(19) United States
(12) Patent Application Publication
(10) Pub. No.: US 2021/0255180 A1
(43) Pub. Date: Aug. 19, 2021

(54) Methods and Kits for Detection of 11-dehydro-thromboxane B2

(71) Applicant: Vascu Technology, Inc., Carlsbad, CA (US)

(72) Inventors: Xiaolin Li, Carlsbad, CA (US); Kenneth A. Browne, Carlsbad, CA (US); Ziye Liu, Carlsbad, CA (US)

(21) Appl. No.: 17/252,641

(22) PCT Filed: Jun. 25, 2019
(86) PCT No.: PCT/US19/38994

§ 371 Date: Dec. 15, 2020

Related U.S. Application Data
(60) Provisional application No. 62/689,426, filed on Jun. 25, 2018.

(51) Int. Cl. G01N 33/558; G01N 33/70; G01N 33/94

(52) U.S. Cl. CPC G01N 33/558; G01N 33/9486; G01N 33/70

(57) ABSTRACT

Methods, compositions and kits for quantitatively determining specified amounts of 11dhTxB2 in microliter to milliliter quantities of a given sample, wherein, the sample is a biological fluid. Specifically, the biological fluid is a quantity of 1 ml or less of urine from a human subject. The methods may be in the form of consolidated assays that can be run in a high throughput, automation format, such as an enzyme-linked immunosorbent assay (ELISA). Further, the ELISA may be modified into a chemiluminescence assay in order to increase sensitivity and linear range and to reduce the reaction time.

### FIELD OF THE INVENTION

This application relates generally to the field of diagnostic devices for determining levels of certain compositions in biological samples. Specifically, the relevant field includes methods, compositions and kits for quantitatively assessing levels of metabolites in biological fluids from human subjects.

### BACKGROUND OF THE INVENTION

Aspirin (also known as acetylsalicylic acid or ASA) is a much-studied and well known medication used in the treatment of pain, fever and inflammatory related conditions. Aspirin is also used prophylactically to prevent heart attacks, blood clots and even certain types of cancer. ASA is a nonsteroidal anti-inflammatory drug (NSAID), and while it functions in a similar manner to other NSAIDS, aspirin has been shown to suppress normal functioning of platelets. Aspirin has also been shown to be highly effective in reducing risks associated with ischemia, myocardial infarction and thrombotic disorders, most likely due to aspirin's known effects on platelets.

Aspirin's century-long usage has generated an incredible amount of data in showing that it is among the world's safest drugs for human use. The mechanism of action of ASA was discovered decades ago by showing that aspirin suppressed the production of prostaglandins and thromboxanes (Vane, J R, “Inhibition of prostaglandin synthesis as a mechanism of action for aspirin-like drugs” Nat. New Biol., 231(25):232-5 (1971)). The manner of aspirin's suppression of two such physiologically active lipid compounds having hormone-like effects is due to its irreversible inactivation of the cyclooxygenase (COX) enzyme required for prostaglandin and thromboxane synthesis (Awtry et al., “Aspirin” Circulation, 101(10):1206-18 (2000)). Specifically, ASA acts as an acetylating agent, with an acetyl group covalently attaching to a serine residue at the active site of the COX enzyme, which is how aspirin is distinct from other NSAIDS such as ibuprofen, which is a reversible inhibitor. This reaction is a precursor to the formation of a variety of prostanoids such as thromboxane A2 (TxA2) and associated metabolites, including 11-dehydrothromboxane B2 (11dhTxB2). Due to its inhibition of TxA2 synthesis, aspirin is one of the most effective therapeutic regimens prescribed as antithrombotic medications and has been shown to reduce the risk of associated cardiovascular events, across a broad swath of patients, by more than 25% (ATT Collaboration, “Collaborative meta-analysis of randomised trials of antiplatelet therapy for prevention of death, myocardial infarction, and stroke in high risk patients” BMJ, 324(7329):71-86 (2002)).

Aspirin acts on at least two different types of COX enzymes, known as COX-1 and COX-2. While aspirin irreversibly inhibits COX-1, it has been shown to modify the enzymatic activity of COX-2, suggesting slightly different mechanisms of action against the different COX enzymes. Furthermore, recent studies have shown aspirin has a minimal effect on platelets for some people, suggesting there is type of resistance or insensitivity to aspirin in certain individuals (Krasopoulos et al., “Aspirin “resistance” and risk of cardiovascular morbidity: systematic review and meta-analysis” BMJ, 336(7637):195-8 (2008); Pignatelli et al., “Multiple anti-atherosclerotic treatments impair aspirin compliance: effects on aspirin resistance” J Thromb. Haemost, 6(10):1832-4 (2008)).

The possibility of resistance to aspirin in an individual will be of critical importance in certain instances, including trauma and/or thrombotic events. Having timely knowledge of an individual's aspirin resistance or level of insensitivity by a caregiver could mean the difference between life and death and, at a minimum, could dictate the type of therapy used based on relevant levels of resistance identified.

Presently, blood based assays exist in order to provide a measurement of possible resistance by assessing platelet aggregation in vitro. However, these results are not specific to aspirin sensitivity and are unable to provide results in a timely fashion at the point of care. Similarly, a quantitative immunoassay is available for metabolite detection in urine requiring the use of a polyclonal antibody to 11dhTxB2. The assay is helpful in that aspirin effectiveness can be determined from a subject's urine, but the polyclonal antibody does not provide highly reproducible or specific results. Moreover, the results would take several hours to acquire, making it virtually useless in many treatment situations involving trauma or thrombotic-related events.

U.S. Pat. No. 8,105,790 to Geske et al. details methods and kits for detection of certain TxA2 metabolites using monoclonal antibodies specific for those metabolites. The disclosure also provides methods for generating monoclonal antibodies from hybridoma cells and using such antibodies in assays lasting 3-5 hours while normalizing against a standard small molecule metabolite for controls. However, the controls are not run in the same assay format as the antibody against the metabolite, and there is no teaching disclosed about how the standard metabolite is measured. Further, the standard metabolite used (creatinine) must be assessed separately, thus relying on a separate assay to normalize the antibody results. Additionally, the time to results of 3-5 hours cannot be used in an environment relying on a more rapid answer to the question of aspirin resistance in an individual.

U.S. Pat. No. 6,994,983 to Ens also describes certain kits for determination of certain metabolites in order to optimize aspirin dosage amounts. However, the disclosure primarily focuses on colorimetric assays in order to determine antibody-metabolite complex formation and is silent with respect to how to test using a standard metabolite, especially at the point of care.

There remains a need in the art for assessing a specific patient's resistance to aspirin quantitatively and reliably and rapidly in order to guide treatment decision-making by a health care professional at the point of care.

### SUMMARY OF THE INVENTION

The present invention provides for methods, compositions and kits for quantitatively determining specified amounts of 11dhTxB2 in microliter to milliliter quantities of a given sample. Preferably, the sample is a biological fluid. More preferably, the biological fluid is a quantity of 1 ml or less of urine from a human subject. In a preferred embodiment, the methods described herein may be in the form of consolidated assays that can be run in a high throughput, automation format, such as an enzyme-linked immunosorbent assay (ELISA). In an alternative embodiment, the ELISA may be modified into a chemiluminescence assay in order to increase sensitivity and linear range and to reduce the reaction time. In an alternative embodiment, the methods of the present invention describe assays that can be administered at the point of care, such as a quantitative lateral flow assay. Each of the alternative embodiments of the methods of the present invention provide a means of independent testing methodologies in order to determine the effectiveness of aspirin within a human subject.

In one aspect, the present invention describes methods and kits wherein the determination of 11dhTxB2 levels in biological samples are run together with internal control analytes for urine volume in the same assay format.

In another aspect, the present invention provides for kits comprising assay formats to test microliter to milliliter urine volumes from a human subject in order to identify specified levels of 11dhTxB2 using in-assay controls comprising creatinine levels from the same samples.

Alternatively, the present invention provides for a method of testing at least one biological sample for the presence and specified amount of TxA2 metabolites by an assay selected from the group consisting of enzyme-linked immunosorbent assay and lateral flow assay. Preferably, the specificity of the method of testing results in specific binding of certain TxA2 metabolites while being unreactive to other TxA2 metabolites.

These features, as well as various alternative embodiments, will be apparent from a reading of the following detailed description of the present invention.

### BRIEF DESCRIPTION OF THE DRAWINGS

The novel features of the present invention are set forth herein embodied in the form of the claims of the invention. Features and advantages of the present invention may be best understood by reference to the following detailed description of the invention, setting forth illustrative embodiments and preferred features of the invention, as well as the accompanying drawings, of which:

FIG. 1 shows the establishment of a standard curve in an ELISA format to quantify 11dhTxB2.

FIG. 2 shows the specificity of detecting one particular metabolite (11dhTxB2) in favor of another, chemically similar metabolite (TxB2).

FIG. 3 shows an example of the lateral flow assay embodiment of the present invention, where the clinical sample is mixed with the relevant tracers and loaded at one end of a membrane strip, with the sample moving in a unidirectional manner over the areas coated with capture antibodies.

FIG. 4 shows the chemiluminescence immunoassay of the present invention measuring 11dhTxB2. The assay detects 11dhTxB2 tracer activity and is quantitatively inhibited by free 11dhTxB2.

FIG. 5 depicts the reduction of time period associated with the chemiluminescence immunoassay of the present invention. The immunoassay measuring 11dhTxB2 can be completed in 70 minutes and in 20 minutes, with the resulting, comparative data shown across both time periods.

FIG. 6 shows the chemiluminescence assay of the present invention measuring creatinine. It covers a linear range of 0.1-100 mM creatinine and takes 20 minutes to complete.

FIG. 7 shows aspirin effectiveness as measured by monitoring normalized 11dhTxB2 (pg/mg creatinine) in urine samples with chemiluminescence assays. Day-1/Day-2 samples are un-treated while Day-3/Day-4 samples are treated by aspirin.

### DETAILED DESCRIPTION OF THE INVENTION

Described herein are methods, compositions and kits for quantitatively determining levels of certain TxA2 metabolite levels in biological samples provided by human subjects. Preferably, the certain TxA2 metabolite to be measured in the biological sample is 11dhTxB2. More preferably, the biological sample is urine.

In some embodiments, the methods of the present invention further comprise at least one normalization element that is present in the same testing format as the quantitative determination of certain TxA2 metabolites. Preferably, the assay methods and kits of the present invention provide a normalization element for urinary dilution by dividing TxA2 metabolite concentrations by creatinine concentrations in the same assay format.

The methods and kits of the present invention comprise an ELISA format for testing specific TxA2 metabolite levels in a urine sample from a human subject in 2 hours or less. Alternatively, the ELISA format adapts chemiluminescent substrates or tracers, which increases sensitivity and linear range of the assay and reduces reaction time. Optionally, the present invention provides an alternative testing embodiment that comprises a lateral flow assay that is conducted at the point of care and may be completed in 15 minutes or less.

Preferably, the ELISA format of the present invention comprises monoclonal antibodies having affinity for and high specific binding characteristics to 11dhTxB2. The monoclonal antibodies described herein may be selectively produced by immunizing an animal selected from the group consisting of human, mouse, rat, horse, rabbit, goat, sheep, chicken, camel or other appropriate animal with an antigen having an epitope similar to or in common with a TxA2 metabolite. Preferably, the TxA2 metabolite is 11dhTxB2.

The following examples are meant to illustrate the present invention and are not meant to be limiting in any manner.

### Examples

I. ELISA Competition Assay to Detect 11dhTxB2

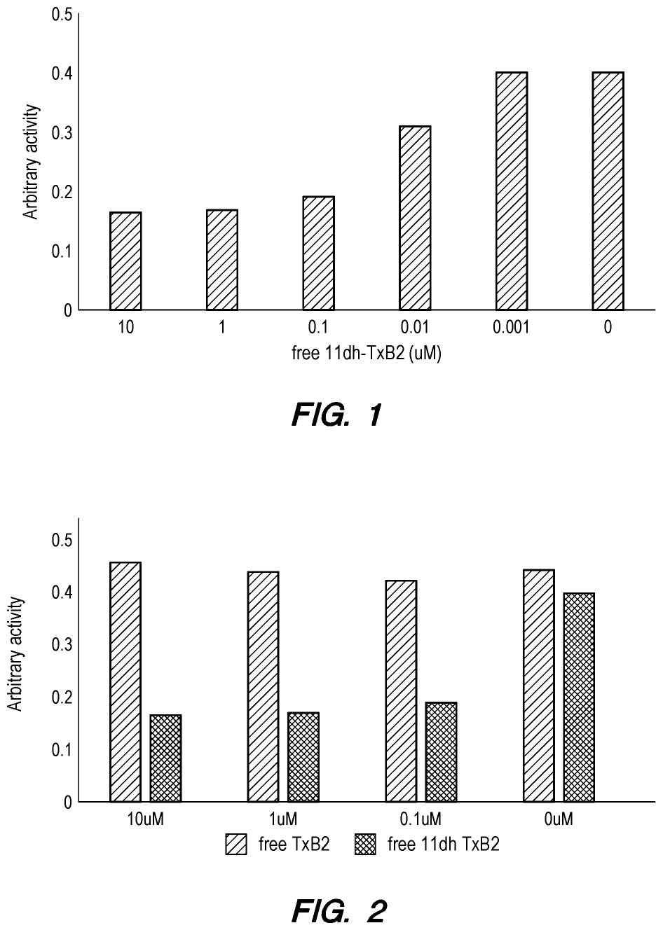

As shown in FIG. 1, the present invention provided for a quantitative measurement of specific metabolite levels using an ELISA format. The ELISA platform was constructed with goat-anti-mouse antibodies coated in a multi-well plate or in multiple tubes.

After the assay was initiated, the coated goat-anti-mouse antibody captured mouse anti-11dhTxB2 antibody, and anti-11dhTxB2 antibody captured an 11dhTxB2 tracer (11dhTxB2 conjugated with alkaline phosphatase) to create readout activity.

Addition of free 11dhTxB2 inhibited this activity in a dose-dependent manner (FIG. 1). The free 11dhTxB2 was utilized to establish a standard curve so the assay was performed as a quantitative metric of binding activity. The free 11dhTxB2 can optionally be clinical samples to be measured by the assay in a quantitative manner.

II. Binding Specificity of ELISA Based Assay for 11dhTxB2

The ELISA based assay of the present invention provided great specificity when detecting the desired target molecule of interest, namely, 11dhTxB2.

To test the specificity of this binding, thromboxane B2 (TxB2) was examined to assess the binding activity in the ELISA format (FIG. 2). TxB2 is a compound that is chemically similar to, but distinct from, 11dhTxB2.

As shown in FIG. 2, TxB2 failed to inhibit the activity of the 11dhTxB2 tracer, confirming the highly specific testing format of the ELISA-based embodiment of the present invention.

### III. ELISA Competition Assay to Detect Creatinine

In order to normalize the activity values determined in the ELISA format, controls are established to compare values across different samples. Accordingly, a creatinine ELISA format is created as described similar to the above Examples.

The creatinine ELISA will be performed not only simultaneously, but in the same assay on the same sample, as the 11dhTxB2 ELISA. In this way, the present invention limits any variables that could skew results across different assays over time. While the creatinine ELISA may not necessarily be performed in the same well as the 11dhTxB2 ELISA, the controls will be run on the same multi-well plate, resulting in improved reliability across testing of samples and reducing time until the two results have been integrated.

The creatinine ELISA utilizes goat-anti-mouse antibody coated on a multi-well polystyrene plate. Once the assay has been initiated, the well's coated goat-anti-mouse antibody will capture mouse anti-creatinine antibody, and anti-creatinine antibody captures a creatinine tracer. Preferably, the -creatinine tracer comprises an anti-creatinine antibody conjugated to alkaline phosphatase, or similar enzyme, which enables the readout activity and measures binding parameters. Addition of free creatinine is expected to inhibit this activity in a dose dependent manner, therefore providing highly reproducible results for standard curve analysis.

The present invention further provides specialized reagents relating to the creatinine ELISA format comprising anti-creatinine antibodies and at least one variation of creatinine tracer. These reagents are utilized in the ELISA format and overcome known problems in the state of the art with respect to antibody and tracer development around the creatinine molecule.

Alternatively, instead of using anti-creatinine antibodies, synthetic antibodies are used to detect creatinine in an immune-like assay format. The synthetic antibodies can be recombinant antibodies, antibody fragments, aptamers, and non-immunoglobulin scaffolds.

Alternatively, instead of using anti-creatinine antibodies or synthetic antibodies, an enzyme that is specific for creatinine binds this metabolite, converts creatinine into one or more different metabolites which can then be detected either in an immunoassay or enzyme assay. Exemplary enzymes include creatinine deiminase to generate N-methylhydantoin and ammonia, and creatinine amidohydrolase to generate creatine.

IV. Lateral Flow Assay to Detect 11dhTxB2 and Creatinine

In an alternative embodiment, the present invention provides for a quantitative lateral flow assay specifically designed to detect 11dhTxB2 and creatinine in the same assay within a matter of minutes based on microliter quantities of biological fluid.

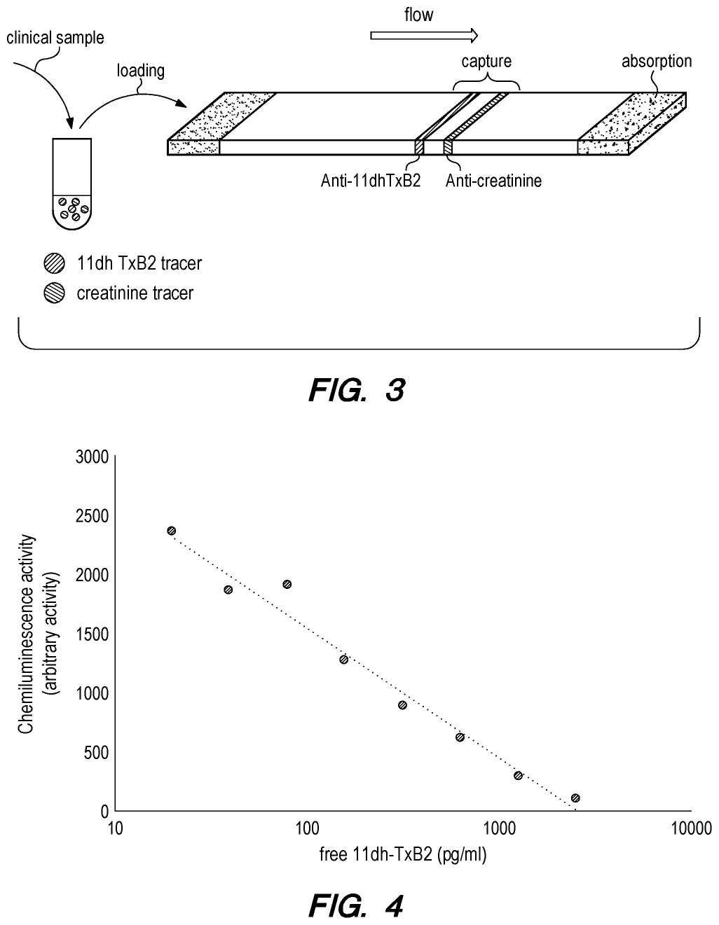

As shown in FIG. 3, a quantitative lateral flow assay is developed in order to detect 11dhTxB2 and creatinine levels in the same assay format. This point of care assay format can be completed in 15 minutes or less, with real time results capable of transmission to the treating physician or clinician.

The lateral flow assay utilizes similar reagents developed for the corresponding ELISA format, namely, the anti-11dhTxB2 and anti-creatinine antibodies. The assay setup includes two different capturing antibodies coated as stripes on a nitrocellulose membrane strip of desired pore sizes. The two different capturing antibodies are captured or embedded into a first capture stripe and a second capture stripe, respectively. The first capture stripe comprises anti-11dhTxB2 antibodies and the second capture stripe comprises anti-creatinine antibodies. Alternatively, the first capture stripe comprises anti-creatinine antibodies and the second capture stripe comprises anti-11dhTxB2 antibodies. Alternatively, the assay setup includes a third antibody coated as a third stripe for use as a control.

A sample is prepared comprising a predetermined amount of two different tracers, namely, 11dhTxB2 tracer and creatinine tracer, which is then mixed together with a clinical sample from a human subject comprising a microliter quantity of a biological fluid. Each tracer comprises magnetic or color-coded beads for use in the lateral flow assay of the present invention. In one embodiment, the tracers in the sample are prepared to covalently link the 11dhTxB2 and creatinine molecule to different sets of magnetic beads (e.g., by size, weight, color (absorbance, reflectance, fluorescence), magnetic parameter, or combinations of these). The sample-tracer mixture is loaded onto the membrane strip and, once the lateral flow assay is initiated, the sample and bead-bound tracers migrate in a unilateral direction down the membrane (FIG. 3). Once the sample and bead-bound tracers cross the first capture stripe and second capture stripe, only the tracer will be captured, with the free molecules in the sample competing with the tracer and thereby reducing the signal of captured, bead-bound tracer in a dose-dependent fashion. The activity is quantitatively measured by a property reading instrument upon loading of the membrane into a cassette for insertion into a reader capable of measuring the requisite parameters.

V. Chemiluminescence Assay to Detect 11dhTxB2

A chemiluminescence immunoassay (CLIA) was developed to detect 11dhTxB2 (FIG. 4). This CLIA had an antibody-based immunoassay principle similar to ELISA, but it had a higher sensitivity and longer detection range than ELISA. The CLIA was set up with goat-anti-mouse antibody coated on a luminescence plate. After the assay is initiated, the coated goat-anti-mouse antibody captures mouse anti-11dhTxB2 antibody, and anti 11dhTxB2 antibody captures an 11dhTxB2 tracer. Addition of free 11dhTxB2 inhibits tracer activity in a dose-dependent manner (FIG. 4). The captured tracer is incubated with chemiluminescent substrates and is measured in a luminescence reader. The free 11dhTxB2 can be utilized as reference materials in order to establish a standard curve, thus enabling the assay to be performed as a quantitative assay. The free 11dhTxB2 can also be clinical samples to be measured against the known standards in the assay in a quantitative manner.

VI. Detection of 11dhTxB2 by CLIA in a Rapid Manner

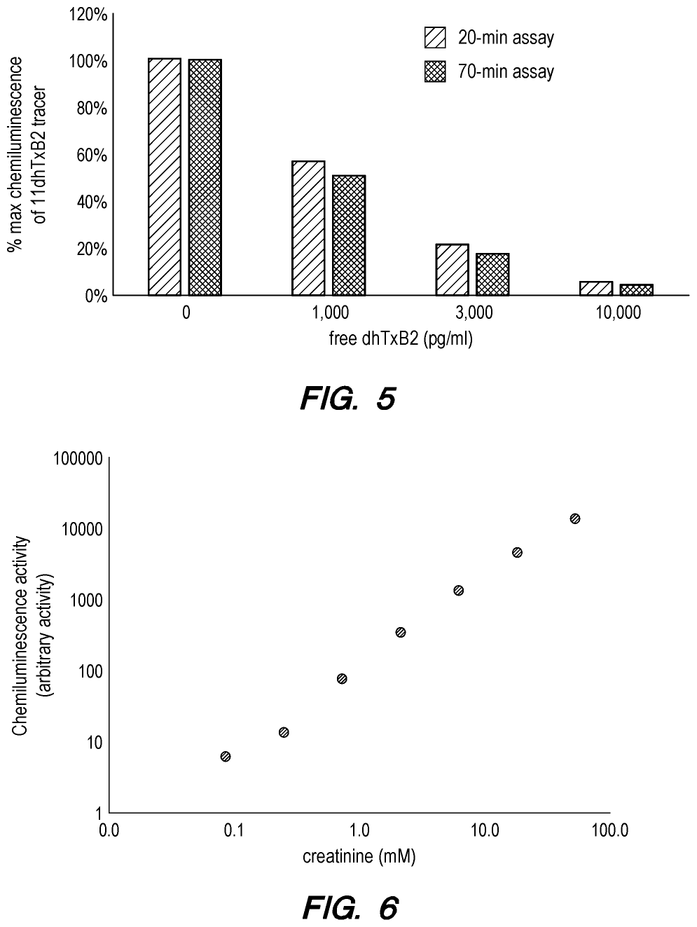

To date, the best ELISA assay for 11dhTxB2 needs at least 2.5 hours incubation time, and the overall assay may need on average 3 hours to complete in order to provide reliable and meaningful results. Compared to the ELISA methods known in the art, our CLIA for 11dhTxB2 has a much-accelerated time frame. CLIAs performed in 70 minutes showed significant free 11dhTxB2 dependent competition (FIG. 5). Assay time longer than 70 minutes do not show further free 11dhTxB2-dependent competition. The 70-minute assay time includes all necessary incubation time, which is equivalent to 2.5 hours incubation time required in the known ELISA methods of the prior art.

The CLIA study also showed that a 20-minute CLIA displays a similar degree of free 11dhTxB2-dependent competition as the 70-minute CLIA (FIG. 5). The absolute luminescence readings in a 20-minute assay are lower than 70-minute (not shown), but degree of inhibition as the main parameter of the assay remains essentially the same. Both 70-minute and 20-minute assays for 11dhTxB2 represents significant improvements in terms of assay efficiency and identification of relevant activity when compared to the 2.5-hour ELISA method.

As we know, point-of-care assays provide quickness and convenience to clinical professionals. In clinical practice, point of care assays usually require completion time in 10-20 minutes. CLIA for 11dhTxB2 assays of the present invention provides a system to detect presence of 11dhTxB2 in 20 minutes or, at least, less than 30 minutes. Thus, on the aspect of time consumption, it is a good candidate system for point-of-care assays. Such an aspect is truly novel, given that this shortened time scale is incapable of being achieved with any success by the current state of the art methods in ELISA techniques.

### VII. Chemiluminescence Assay for Creatinine

The enzymatic assays described herein are capable of accurately and rapidly measuring creatinine levels in clinical samples. They utilize a series of enzymes including creatinine amidohydrolase, creatine amidohydrolase, sarcosine oxidase that convert creatinine into H₂O₂ (as a tracer that it proportional to creatinine concentration), and eventually H₂O₂ level will be measured by a chemiluminescent substrate.

The creatinine assays available today predominantly take colorimetric measurements. Due to the intrinsic limits associated with such colorimetric elements, its detection range is about 15 to 20-fold. For usual clinical creatinine concentrations (at 1-30 mM), the current state of the art assays require dilution steps for samples with high concentrations of creatinine. For low level creatinine concentrations, although no dilutions are needed, colorimetric assays could have difficulty to detect or detect with reasonable variations.

The chemiluminescence creatinine assays of the present invention provide wide detection ranges that cover from about 0.1-100 mM (FIG. 6). This 1000-fold detection range not only includes the usual creatinine range in urine samples at 1-30 mM, but also readily covers most outliers that fall outside such ranges. Outliers such as low-level creatinine at 0.5-1 mM could happen when a patient consumed too much fluid prior to the sample draw. The chemiluminescence creatinine assay dosing requires no further dilution of samples for high levels of creatinine, and is simultaneously capable of detecting low level creatinine (as low as 0.1 mM). The assay time is 20 minutes, which qualifies such assay as an ideal system to measure creatinine in a point-of-care device.

VIII. Normalized Level of 11dhTxB2 as Indicator of Aspirin Effects

Chemiluminescence is an assay platform on which both 11dhTxB2 and its internal control can be measured efficiently, simultaneously and quantitatively. We set out to test its ability to examine clinical samples and monitor aspirin effects on 11dhTxB2. In this study, urine samples from healthy volunteers were collected once a day for 4 consecutive days. A dose of aspirin (325 mg) was taken by each donor after Day-2 sample collections, thus Day-1 and Day-2 samples were un-treated aspirin while Day-3 and Day-4 samples were after treatment with aspirin. After all samples were collected, 11dhTxB2 and creatinine were measured in the chemiluminescence assay of the present invention.

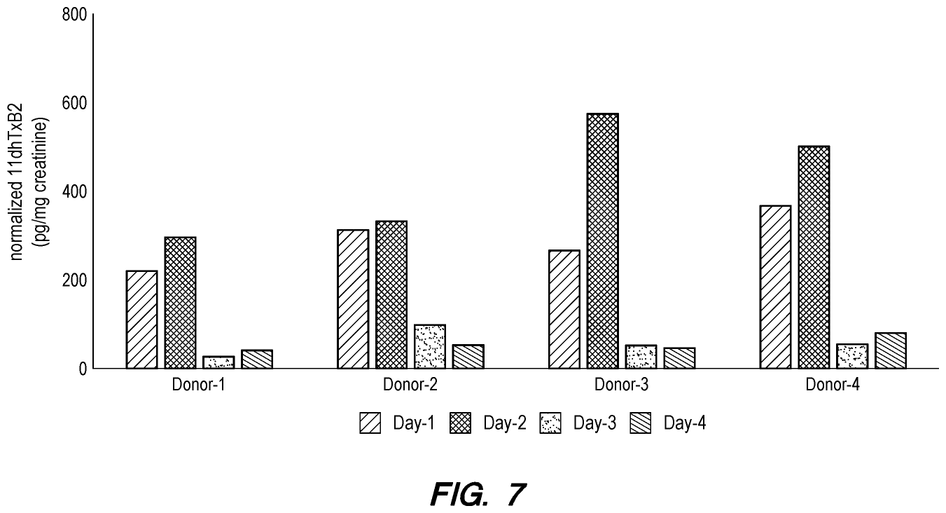

The results clearly indicate that aspirin reduces the levels of normalized 11dhTxB2 in urine by 70% or more (FIG. 7). This is consistent with the established effects of aspirin. Our results indicate that chemiluminescence assays provide effective, sensitive and efficient measurements for 11dhTxB2 and for creatinine in urine samples.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features. As used in this specification and in the appended claims, the singular forms include the plural forms. For example, the terms “a,” “an,” and “the” include plural references unless the content clearly dictates otherwise. Additionally, the term “at least” preceding a series of elements is to be understood as referring to every element in the series. The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein. In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should, therefore, be determined not with reference to the above description, but should instead be determined with reference to the appended claims, along with the full scope of equivalents to which such claims are entitled. Those skilled in the art will recognize, or will be able to ascertain using no more than routine experimentation, many equivalents to the specific embodiments of the invention described. Such equivalents are intended to be encompassed by the following claims.

What is claimed is:

1. A lateral flow assay for the identification and quantification of aspirin sensitivity in an individual, comprising:
(a) providing a nitrocellulose membrane strip;
(b) providing at least two different capturing antibodies coated on the nitrocellulose membrane strip;
(c) preparing a sample comprised of a biological sample from the individual mixed with a predetermined amount of at least two different tracers, wherein the at least two different tracers comprise magnetic or color-coded beads for use in the assay;
(d) loading the sample onto the nitrocellulose membrane strip and allowing the sample to migrate in a unilateral direction down the nitrocellulose membrane strip; and
(e) measuring activity of the sample through use of a reader capable of measuring the required parameters.

2. The assay of claim 1, wherein the at least two different capturing antibodies are embedded into at least a first capture stripe and a second capture stripe.

3. The assay of claim 2, wherein the first capture stripe comprises anti-11dhTxB2 antibodies and the second capture stripe comprises anti-creatinine antibodies.

4. The assay of claim 1, wherein the at least two different tracers are 11dhTxB2 and creatinine.

5. The assay of claim 4, wherein the 11dhTxB2 and creatinine tracers are covalently linked to different sets of magnetic beads, wherein each set is characterized by a difference selected from the group consisting of size, weight, color and combinations thereof.

6. A method of testing at least one biological sample for the presence and amount of thromboxane A2 metabolites comprising providing an assay selected from the group consisting of an enzyme linked immunosorbent assay and lateral flow assay; mixing the at least one biological sample with assay specific antibody reagents to form a sample cocktail; mixing the at least one biological sample with assay specific control reagents to form a control cocktail; adding the sample cocktail and control cocktail into the assay; assessing binding results from the sample cocktail against the control cocktail to arrive at the presence and amount of the thromboxane A2 metabolites in the at least one biological sample.

7. The method of claim 6, wherein the at least one biological sample is from a mammal.

8. The method of claim 6, wherein the at least one biological sample is urine or blood.

9. The method of claim 6, wherein the antibody reagents are derived from natural sources.

10. The method of claim 6, wherein the antibody reagents are synthetically derived.

11. The method of claim 6, wherein the presence and amount of the thromboxane A2 metabolites in the at least one biological sample is achieved in less than 30 minutes.

12. The method of claim 6, wherein the lateral flow assay is conducted at a point of care and is completed in 15 minutes or less.

13. A method of identifying and quantifying aspirin sensitivity in an individual comprising providing a biological sample from the individual, providing a chemiluminescence immunoassay capable of detecting 11dhTxB2 amounts across a 1000-fold detection range and testing the biological sample using the chemiluminescence immunoassay.

14. The method of claim 13, wherein an amount of free 11dhTxB2 is added to the immunoassay and used to establish a standard curve for use in comparing against the biological sample test results.

15. A kit for quantitatively determining specified amounts of a metabolite present in a sample, wherein the sample is a biological fluid and consists of a quantity in the microliter to milliliter range, wherein the kit further comprises an assay selected from the group consisting of enzyme-linked immunosorbent assay, chemiluminescence assay and quantitative lateral flow assay, wherein the assay comprises at least one control run in parallel with the sample as tested.

16. The kit of claim 15, wherein the metabolite is 11-dehydrothromboxane B2.

17. The kit of claim 15, wherein the at least one control is creatinine or a metabolite thereof.

18. The kit of claim 15, wherein the assay comprises monoclonal antibodies in order to specifically bind the metabolite present in the sample.

19. The kit of claim 18, wherein the monoclonal antibodies may be selectively produced by immunizing an animal selected from the group consisting of human, mouse, rat, horse, rabbit, goat, sheep, chicken, camel and any other animal with an antigen having an epitope similar to or in common with a thromboxane A2 metabolite.

20. The kit of claim 17, wherein the assay comprises anti-creatinine antibodies to detect creatinine in the assay.

21. The kit of claim 20, wherein the anti-creatinine antibodies are synthetic antibodies selected from the group consisting of recombinant antibodies, antibody fragments, aptamers and non-immunoglobulin scaffolds.

22. The kit of claim 17, wherein the creatinine control is measured by providing at least one enzyme that is specific for creatinine and exposing the at least one enzyme to the assay in order to convert the creatinine into an alternative form capable of being detected by the assay, the at least one enzyme selected from the group consisting of creatinine deiminase and creatinine amidohydrolase.

23. The kit of claim 17, wherein the creatinine control is measured by a chemiluminescence assay.
